(19) United States
(12) Patent Application Publication
(10) Pub. No.: US 2003/0180765 A1
(43) Pub. Date: Sep. 25, 2003

(54) Digital amplification for detection of mismatch repair deficient tumor cells

(75) Inventors: Carlo Giovanni Traverso, Etobicoke (CA); Kenneth W. Kinzler, Bel Air, MD (US); Bert Vogelstein, Baltimore, MD (US)

Correspondence Address: BANNER & WITCOFF, 1001 G STREET N W, SUITE 1100, WASHINGTON, DC, 20001, US

(73) Assignee: The Johns Hopkins University, Baltimore, MD

(21) Appl. No.: 10/349,173

(22) Filed: Jan. 23, 2003

Related U.S. Application Data
(60) Provisional application No. 60/352,869, filed on Feb. 1, 2002.

(51) Int. Cl. C12Q 1/68; C12P 19/34

(52) U.S. Cl. 435/6; 435/91.2

(57) ABSTRACT

The detection of mutations in fecal DNA represents a promising, non-invasive approach for detecting colorectal cancers in average risk populations. One of the first practical applications of this technology involves the examination of microsatellite markers to sporadic cancers with mismatch repair deficiencies. As such cancers nearly always occur in the proximal colon, this test is useful as an adjunct to sigmoidoscopy, which detects only distal colorectal lesions.

The U.S. government retains certain rights in this invention by virtue of its support of the underlying research, supported by grants CA 62924 and CA 43460 from the National Institutes of Health.

### TECHNICAL FIELD OF THE INVENTION

This invention is related to diagnostic genetic analyses. In particular it relates to detection of genetic changes in colorectal cancers.

### BACKGROUND OF THE INVENTION

Colonoscopy, sigmoidoscopy, and double contrast barium enema provide excellent tests for neoplasia but are limited by their invasive nature, requirement for highly trained personnel, and patient compliance.¹ Tests for fecal occult blood (FOBT) are non-invasive and useful, especially as an adjunct to sigmoidoscopy.¹ However, the relatively high false positivity rates and other problems with FOBT have led to a search for more specific non-invasive tests. In this regard, assays for mutations in fecal DNA offer particular promise.² Most previous studies in this area have focused on the more common lesions of the distal colon and rectum (³ and references therein). There is a need in the art for methods for detecting proximal cancers in patients. Proximal cancers should be the most difficult to detect, as they are farthest from the anus.

### SUMMARY OF THE INVENTION

According to one embodiment of the invention a method is provided for detecting proximal colorectal cancers. A test fecal sample isolated from a patient is divided to form a plurality of aliquots. The aliquots comprise on average from 0 to 100 BAT26 alleles. The BAT26 alleles in the aliquots are amplified using a first primer and a second primer to form amplified templates. The amplified templates are themselves amplified using the first primer and a third primer to form amplified subtemplates. The size of the amplified subtemplates of each aliquot is analyzed. An altered size of amplified subtemplates in at least one aliquot indicates a mismatch repair-deficient proximal colorectal cancer in the patient. Altered size is determined relative to size of amplified subtemplate amplified from wild-type BAT26 alleles from a non-cancer patient.

According to another embodiment of the invention a method is provided for screening for proximal and distal colorectal tumors in a patient. A test fecal sample isolated from a patient is divided to form a plurality of aliquots. The aliquots comprise on average from 0 to 100 BAT26 alleles. The BAT26 alleles in the aliquots are amplified using a first primer and a second primer to form amplified templates. The amplified templates are themselves amplified using the first primer and a third primer to form amplified subtemplates. The size of the amplified subtemplates of each aliquot is analyzed. An altered size of amplified subtemplates in at least one aliquot indicates a mismatch repair-deficient proximal colorectal cancer in the patient. Altered size is determined relative to size of amplified subtemplate amplified from wild-type BAT26 alleles from a non-cancer patient. A sigmoidoscopy is performed on the patient to detect distal colorectal tumors.

Also provided by the present invention is a kit comprising a set of primers for performing hemi-nested PCR. A first primer of the set comprises a sequence ₅′-CAGTATATGAAATTGGATATTGCAG-3′ (SEQ ID NO: 1). A second primer of the set comprises a sequence 5′-CTTCTTCAGTATAT GTCAATGAAAAC-3′ (SEQ ID NO: 2). A third primer of the set comprises a sequence 5′-AGCAGTCAGAGCCCTTAACCTTT-3′ (SEQ ID NO: 3).

### BRIEF DESCRIPTION OF THE DRAWINGS

FIGURE: BAT26 Assay. Representative examples of capillary electrophoretograms from a single patient. Capillaries 1 & 2 contained normal BAT26 alleles while capillaries 3 & 4 contained both mutated and normal BAT26 alleles. Capillary 5 contained PCR-amplified DNA from this patient's cancer. Example of wild type and mutant peaks are indicated by green and red arrows, respectively. Seventy-two capillaries were analyzed for each patient following hemi-nested amplification of fecal or tumor DNA with a fluorosceinated primer. The initial amplification was performed with F1 5′-CAGTATATGAAATTGGATATTGCAG-3′ and R1 5′-CTTCTTCAGTATATGTCAATGAAAAC-3′; a small aliquot of the first amplification was used as a template for hemi-nested amplifications with F1 and R2 5′-AGCAGTCAGAGCCCTTAACCTTT-3′.

### DETAILED DESCRIPTION OF THE INVENTION

The method devised by the present inventors involves separately amplifying small numbers of template molecules so that the resultant products have a proportion of the analyte sequence which is detectable by the detection means chosen. At its limit, single template molecules can be amplified so that the products are completely mutant or completely wild-type (WT). The homogeneity of these amplification products makes them trivial to distinguish through existing techniques. BAT26 has been selected as an allele for analysis because it has been found to be a microsatellite marker which is altered in an extremely high proportion of mismatch repair deficient colorectal cancers. Other markers which are similarly high frequency targets of microsatellite instability can be used as well. For example, any of BAT25, D2S123, D5S346, and D17S250, FGA, D18S35, and TP53-DIcan be used.

The method requires analyzing a large number of amplified products simply and reliably. A suitable number of separately amplified products (reactions) ranges from 10 to 150, more preferably 15 to 100, or even more preferably 25 to 80. Larger numbers of reactions analyzed will increase the sensitivity of detection.

The biological sample is diluted to a point at which a practically usable number of the diluted samples contain a proportion of the selected genetic sequence (analyte) relative to total template molecules such that the analyzing technique being used can detect the analyte. A practically usable number of diluted samples will depend on cost of the analysis method. Typically it would be desirable that at least 1/50 of the diluted samples have a detectable proportion of analyte. At least 1/10, ⅕, 3/10, ⅖, ½, ⅗, 7/10, ⅘, or 9/10 of the diluted samples may have a detectable proportion of analyte. The higher the fraction of samples which will provide useful information, the more economical will be the overall assay. Over-dilution will also lead to a loss of economy, as many samples will be analyzed and provide no signal. A particularly preferred degree of dilution is to a point where each of the assay samples has on average 0 to 100 BAT26 templates. More preferably the assay samples or aliquots contain 0 to 50 BAT26 templates. Even more preferably the aliquots contain on average 0 to 20 BAT26 templates. Dilution of a fecal sample can be performed from a more concentrated sample. Alternatively, dilute sources of template nucleic acids can be used, in which case dividing of the sample without dilution can be employed. All of the samples may contain amplifiable template molecules.

Digital amplification can be used to detect mutations such as microsatellite size changes which are present at relatively low levels in the samples to be analyzed. The limit of detection is defined by the number of wells that can be analyzed and the intrinsic mutation rate of the polymerase used for amplification. 384 well PCR plates are commercially available and 1536 well plates are on the horizon, theoretically allowing sensitivities for mutation detection at the ˜0.1% level. The amplification can be performed in microarray format, potentially increasing the sensitivity by another order of magnitude. This sensitivity may ultimately be limited by polymerase errors.

If the allele to be analyzed is transcribed, then amplification can be performed on RT-PCR products generated from RNA templates or on genomic DNA. Methods for generating amplification templates from mRNA are well known in the art and any such method can be employed.

In one preferred embodiment each diluted sample has on average one half a template molecule. This is the same as one half of the diluted samples having one template molecule. This can be empirically determined by amplification. Either the analyte (selected genetic sequence) or the reference genetic sequence can be used for this determination. If the analysis method being used can detect analyte when present at a level of 20%, then one must dilute such that a significant number of diluted assay samples contain more than 20% of analyte. If the analysis method being used requires 100% analyte to detect, then dilution down to the single template molecule level will be required.

The method of the invention requires analysis of a large number of samples to get meaningful results. Preferably at least ten diluted assay samples are amplified and analyzed. More preferably at least 15, 20, 25, 30, 40, 50, 75, 100, 500, or 1000 diluted assay samples are amplified and analyzed. As in any method, the accuracy of the determination will improve as the number of samples increases, up to a point. Because a large number of samples must be analyzed, it is desirable to reduce the manipulative steps, especially sample transfer steps. Thus it is preferred that the steps of amplifying and analyzing are performed in the same receptacle. This makes the method an in situ, or “one-pot” method.

Biological samples which can be used as the starting material for the analyses may be from any tissue or body sample from which DNA or mRNA can be isolated. Preferred sources include stool, blood, and lymph nodes. Preferably the biological sample is a cell-free lysate.

The fraction of aliquots with an altered size of amplified BAT26 subtemplate relative to aliquots with only wild-type size amplified BAT26 subtemplate can be determined. Fecal samples which provide a fraction of between 0.01 to 0.11 indicate a sporadic cancer.

Any primers can be used for amplifying the BAT26 allele. Particularly preferred primers for amplifying the BAT26 allele include 5′-CAGTATATGAAATTGGATATTGCAG-3′ (SEQ ID NO: 1), 5′-CTTCTTCAGTATATGTCAATGAAAAC-3′ (SEQ ID NO: 2), and 5′-AGCAGTCAGAGCCCTTAACCTTT-3′ (SEQ ID NO: 3). The primers can be labeled with any detectable label known in the art. Particularly preferred is fluorescein, but other labels which are highly detectable and convenient can be used.

The above disclosure generally describes the present invention. A more complete understanding can be obtained by reference to the following specific examples which are provided herein for purposes of illustration only, and are not intended to limit the scope of the invention.

### EXAMPLE 1

A total of 134 stool samples for which informed consent had been obtained were analyzed, derived from 46 patients with cancers of the proximal colon (i.e. between the cecum and hepatic flexure), 19 patients with proximal adenomas, and 69 patients who were colonoscopically normal. The reasons for performing colonoscopy in the latter group included positive fecal occult blood tests, rectal bleeding, or personal or family history of colorectal neoplasia.

Stool samples were obtained prior to beginning laxative treatments to prepare for surgery or colonoscopy. They were immediately stored at −20° C. and a randomly chosen 1 to 10 g aliquot was transferred to −80° C. within 48 hours. None of the patients had familial adenomatous polyposis or hereditary non-polyposis colon cancer. We used the BAT26 marker as an indicator of microsatellite instability, as the mononucleotide tract in BAT26 has been shown to be altered in nearly all mismatch-deficient tumors.⁴ DNA was purified from stool using hybrid capture with oligonucleotides specific to the BAT26 locus. A Digital PCR based method⁵ was then used to analyze the concentration and mutational fraction of each fecal DNA sample. In brief, limiting dilution of the DNA was employed to determine the concentration of BAT26 genes in each sample. For this determination, fecal DNA was used as a template for PCR with fluorescein-labeled primers, and the products separated through capillary electrophoresis. Then DNA samples were diluted so that ˜7 template molecules were present in each well. By analyzing only a small number of template molecules per reaction, the signal to noise ratio (mutant/wild type) of the BAT26 sequences was maximized. Through analysis of 72 wells per patient, we were able to assess ˜500 template molecules per assay. This analysis was robotically automated, and the PCR products of all 72 wells analyzed in parallel in a 192 capillary instrument.

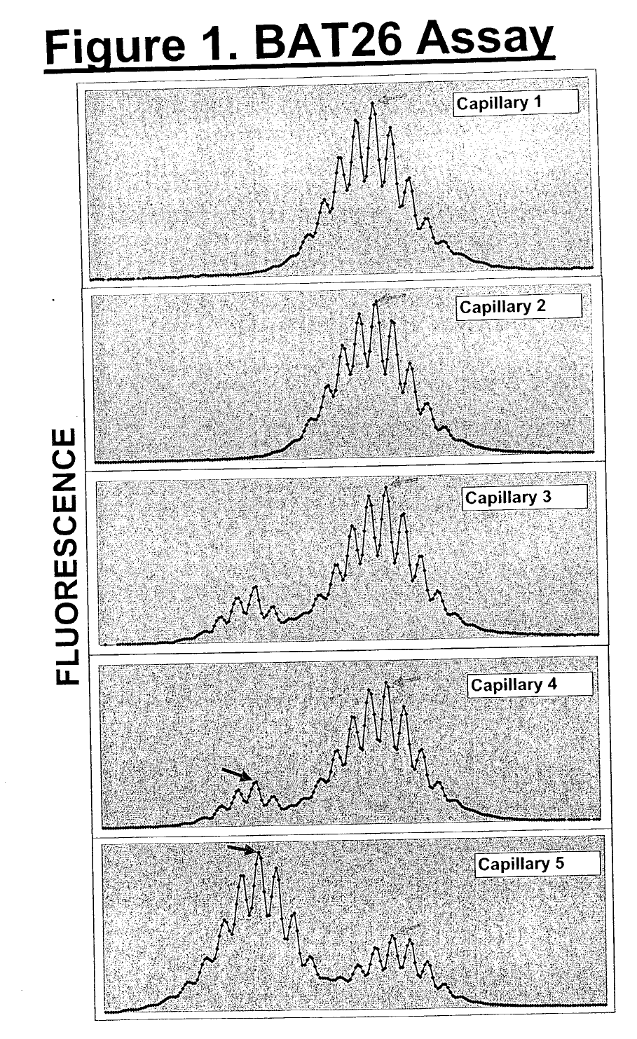

The fecal DNA analyses were done in a blinded fashion. Of 134 samples analyzed, 17 were found to have BAT26 alterations. Examples of the results from this assay are shown in FIG. 1. All 17 fecal DNA samples yielding a positive BAT26 test were subsequently determined to have been derived from a patient with colorectal cancer (Table 1).

Among the cancer patients containing proximal lesions, the clinical sensitivity of the BAT26 fecal DNA test was 37% (17 of 46, 95% confidence interval 23% to 52%), with no positives among 69 individuals with normal colonoscopies or among 19 individuals with adenomas. The specificity was therefore 100%, with 95% confidence interval 95% to 100%. To determine the concordance of BAT26 alterations between fecal DNA and tumors, we microdissected neoplastic lesions from paraffin-embedded specimens of all 65 tumors (46 cancers plus 19 adenomas). DNA of adequate quality was recovered from 57 lesions, and 18 cases with BAT26 alterations were observed, all among cancers. Seventeen of these 18 cases corresponded to those with positive fecal tests, and in each of these cases, the size of the BAT26 alteration in stool and fecal DNA was identical (FIG. 1).

The results recorded above have several important implications for fecal DNA testing. First, the results provide compelling evidence that mutations in stool can be used to identify patients with cancer. The fact that seventeen of the 18 cases with BAT26 mutations in their tumors gave rise to a positive fecal DNA test, coupled with the zero false positive rate, was of particular note. Second, the results show that proximal cancers do not represent a barrier to fecal DNA analysis. Third, it was clear that small aliquots of stool, rather than whole stools, could be effectively analyzed, facilitating collection and storage of specimens for analysis. Finally, the fraction of mutant DNA molecules in fecal DNA was found to range from 1.1% to 10.6%. Thus, techniques to assess fecal DNA mutations need be no more sensitive than this to detect the great majority of mutations. In the one sample that was a false negative, increasing the potential sensitivity five-fold by analyzing an additional 2000 BAT26 genes in fecal DNA did not result in detection of the mutation.

One practical application of these results involves combination of BAT26 with sigmoidoscopy. Cost-effectiveness modeling has indicated that sigmoidoscopy combined with unhydrated FOBT can be more effective than colonoscopy for CRC screening.¹ The sensitivity of the BAT26 assay is similar to that of the unrehydrated FOBT but is more expensive. This cost disadvantage is counterbalanced by the fact that the BAT26 test appears to be considerably more specific, thereby precluding the need for follow-up colonoscopies in a substantial fraction of patients with false positive FOBTs. Furthermore, the BAT26 test does not require patients to change their dietary habits prior to testing, nor to provide multiple fecal samples, potentially increasing compliance.

TABLE 1
Results of analysis of fecal DNA for BAT26 alterations

| | | Total | | |
|---|---|---|---|---|
| | | number of | Pos. BAT26 | Neg. Bat26 in |
| Patient Group | | patients | in fecal DNA | fecal DNA |
| No neoplasia | | 69 | 0 | 69 |
| With Adenoma | | 19 | 0 | 19 |
| | <1 cm | 14 | 0 | 14 |
| | ≧1 cm | 5 | 0 | 5 |
| With Cancer | | 46 | 17 | 29 |
| | Dukes' A | 5 | 1 | 4 |
| | Dukes' B | 22 | 11 | 11 |
| | Dukes' C | 11 | 4 | 7 |
| | Dukes' D | 8 | 1 | 7 |

### EXAMPLE 2

PCR

Each reaction contained 1×PCR Buffer (Invitrogen, Carlsbad, Calif.), 0.9 μM oligonucleotides F1 and R1, and 0.005 U per microliter Platinum Taq DNA Polymerase High Fidelity (Invitrogen, Carlsbad, Calif.). A single PCR mix was prepared for each stool sample and the mix aliquotted to 72 wells, representing 6 rows of 12 wells of a standard 96-well PCR plate. Each well contained approximately 7 BAT26 templates distributed in a Poisson distribution. After an initial denaturation at 94° C. for 2 minutes, amplifications were performed as follows: 60 cycles of: 94° C. for 15 seconds, 56° C. for 15 seconds, 70° C. for 15 seconds. One μL of the reaction was added to a 10-μL PCR reaction of the same makeup as the one described above except that primers F1 and R2 were used. Following a 2 minute denaturation step at 94° C., the reaction was cycled for 15 cycles of: 94° C. for 15 seconds, 56° C. for 15 seconds, 70° C. for 15 seconds. Primer sequences were:

| | |
|---|---|
| F1 | 5′-CAGTATATGAAATTGGATATTGCAG-3′; |
| R1 | 5′-CTTCTTCAGTATATGTCAATGAAAAC-3′; |
| R2 | Fluorescein-5′-AGCAGTCAGAGCCCTTAACCTTT′-3′. |

Capillary Electrophoresis

PCR reactions were analyzed by adding 1 μL to 9 μL of formamide. Samples were analyzed on a SCE-9610 192-well capillary electrophoresis system (SpectruMedix Corporation, State College, Pa.).

### REFERENCES

The disclosures of each of the following are incorporated herein by reference for all purposes.

1. Frazier A L, Colditz G A, Fuchs C S, Kuntz K M. Cost-effectiveness of screening for colorectal cancer in the general population. Jama 2000; 284:1954-61.

2. Ahlquist D A, Shuber A P. Stool screening for colorectal cancer: evolution from occult blood to molecular markers. Clin Chim Acta 2002; 315:157-68.

3. Traverso G, Shuber A, Levin B, et al. Detection of APC mutations in fecal DNA from patients with colorectal tumors. N Engl J Med 2002; 346:311-20.

4. Loukola A, Eklin K, Laiho P, et al. Microsatellite marker analysis in screening for hereditary nonpolyposis colorectal cancer (HNPCC). Cancer Res 2001; 61:4545-9.

5. Vogelstein B, Kinzler K W. Digital PCR. Proc Natl Acad Sci U S A 1999; 96:9236-41.

1. A method for detecting proximal colorectal cancers, comprising:
dividing a test fecal sample isolated from a patient to form a plurality of aliquots, wherein said aliquots comprise on average from 0 to 100 BAT26 alleles;
amplifying said BAT26 alleles in said aliquots using a first primer and a second primer to form amplified templates;
amplifying the amplified templates using the first primer and a third primer to form amplified subtemplates;
analyzing size of the amplified subtemplates of each aliquot, wherein an altered size of amplified subtemplates in at least one aliquot indicates a mismatch repair-deficient proximal colorectal cancer in the patient, wherein an altered size is determined relative to size of amplified subtemplate amplified from wild-type BAT26 alleles from a non-cancer patient.

2. The method of claim 1 further comprising determining a fraction of aliquots with an altered size of amplified subtemplate relative to aliquots with only wild-type size amplified subtemplate, said aliquots having been divided from a single test fecal sample, wherein a fraction of 0.01 to 0.11 indicates a sporadic cancer.

3. The method of claim 1 wherein the first primer is 5′-CAGTATATGAAATTGGATATTGCAG-3′ (SEQ ID NO: 1).

4. The method of claim 1 wherein the second primer is 5′-CTTCTTCAGTATATGTCAATGAAAAC-3′ (SEQ ID NO: 2).

5. The method of claim 1 wherein the third primer is 5′-AGCAGTCAGAGCCCTTAACCTTT-3′ (SEQ ID NO: 3).

6. The method of claim 1 wherein the first primer is 5′-CAGTATATGAAATTGGATATTGCAG-3′ (SEQ ID NO: 1) and the second primer is 5′-CTTCTTCAGTATATGTCAATGAAAAC-3′ (SEQ ID NO: 2) and the third primer is 5′-AGCAGTCAGAGCCCTTAACCTTT-3′ (SEQ ID NO: 3).

7. The method of claim 1 wherein the third primer is labeled.

8. The method of claim 1 wherein the third primer is labeled with fluorescein.

9. The method of claim 6 wherein the third primer is labeled.

10. The method of claim 6 wherein the third primer is labeled with fluorescein.

11. The method of claim 7 wherein the third primer is labeled.

12. The method of claim 7 wherein the third primer is labeled with fluorescein.

13. The method of claim 1 wherein the step of dividing is performed by dilution.

14. The method of claim 1 wherein BAT26 alleles in 10 to 150 aliquots are amplified and analyzed.

15. The method of claim 1 wherein BAT26 alleles in 15 to 100 aliquots are amplified and analyzed.

16. The method of claim 1 wherein BAT26 alleles in 25 to 80 aliquots are amplified and analyzed.

17. The method of claim 1 wherein the aliquots comprises on average from 0 to 20 BAT26 alleles.

18. The method of claim 1 wherein said aliquots comprise on average from 0.1 to 10 BAT26 alleles.

19. A method for screening for proximal and distal colorectal tumors in a patient, comprising:
performing the method of claim 1 to detect proximal colorectal tumors and performing a sigmoidoscopy to detect distal colorectal tumors.

20. A kit comprising a set of primers for performing hemi-nested PCR, said set comprising:
first primer 5′-CAGTATATGAAATTGGATATTGCAG-3′ (SEQ ID NO: 1) and second primer 5′-CTTCTTCAGTATAT GTCAATGAAAAC-3′ (SEQ ID NO: 2) and third primer 5′-AGCAGTCAGAGCCCTTAACCTTT-3′ (SEQ ID NO: 3).
